SEQUENCE LISTING (20 sequences)

SEQ ID NO: 1 - DNA, 1662 bp, Artificial Sequence
  Fungi
atgtcagaacctctaccccctaaagaaggggaaccaaggccacagaaggaagaaagtcaaaatgacacgctcgaagcgactgagtccaagtcccagcacatcacaggcctcaagctcgggctggtggttgcttcagttactttcgtagcatttttgatgctccttgatatgtccattatcgtcacggcaatcccacatatcacaagcgagttccactctctgaacgatgtagggtggtacggcagtgcttatcttctggctaactgtgctctccagcccctggccggtaaattgtatacactcttgggcttgaagtacactttctttgccttcctctgtatttttgaactaggctcggtgctatgcggtgccgcaagatcttccaccatgttgattgttgggcgggccgttgctggaatgggaggctcaggtcttgtcaacggagccctcacaatcctctcaacagctgctcctaagcacaagcaaccagttttgattggagtgatgatgggtcttagtcagattgccattgtctgtggaccactgctcggaggtgctttcactcaacacgccacttggcgatggtgcttttatatcaatctccccatcggcgctgtcgctgcattcctccttctcgtcatcaccatacccgaccgaatttcatccacggacagcgaactctcgaccgacaaaccaatggccaacataaaatccacacttcgcaaactggaccttgtaggctttgtggtctttgcagccttcgcaaccatgatttccctcgcactagaatggggagggtcgacctacacctggcgaagttccgtcatcatcggcctgttctgtggcggagggtttgctctgattgcgttcgtgctatgggagcgtcatgttggcgatgctgttgccatgattcctggctcagtggctggtaaacgacaagtgtggtgctcttgtttatttatgggctttttctctggctccttgcttgtcttttcctactatctaccgatctacttccaggctgtgaaggatgtttctcccacactgagtggtgtgtacatgttgcctggaattcttggacaagtcattatggctatggtttctggcttcgcaattggaaagacaggatactatttgccttgggccctaggcagcgctgttctcgtcgccataggcgcaggtctggtatccaccttccagccccatacatcaactgtgaaatgggtcatgtaccaatttatcgcgggcttcggtcgtggttgtggcatgcaaacgcctatcatcgccatccaaagcacgctttcgcccgaacaaggtgccctcggaatttctctcgccgtgtttggacagacgtttggaggatcgctcttcctggactttgctaaccttgtctttgggtccggtttgagaactggcctgagcaagtatgcgcccactgtcgacacgcaggccgtgacggcagcaggggcgactggcttcagagatgtggtcagcaagaataaccttccaggggttgtaaaagcttacagtctcgccgttgatcatactttttacttagcagtgggagctacggcgtgcacgtttgtgtttgcctttggaatgggatggcggaagattgcaaccaaaaacgacacccgggctgtgcctgagactgatgcttga

SEQ ID NO: 2 - DNA, 1662 bp, Artificial Sequence
  Fungi
atgagtgaaccattaccaccaaaggaaggggaaccaagaccacaaaaagaggaatctcaaaatgacacactggaagctaccgaatcaaaatcccagcacataaccggccttaaacttgggttggtagtagcatctgttaccttcgttgctttccttatgttactagatatgtccatcattgtaacagctattcctcacattacatcagaattccattcattgaatgatgttggctggtacggtagtgcttatcttttagctaattgtgccctgcaaccattagctggcaaattgtatactttgctggggttaaagtacacctttttcgcctttttgtgtatcttcgaattaggatctgtcctatgtggtgctgctcgtagttctactatgttaatcgtcgggcgtgccgtggccggtatgggcggatccggtttggtcaatggtgctctgacaatcttgtcaactgcagcccctaaacataagcagcctgttctaataggtgttatgatgggtctatcccaaattgctattgtgtgtggcccattgcttggcggtgcttttactcaacatgccacatggagatggtgtttctacatcaatctgccaattggtgccgttgccgcttttctactattggtgattacaattccagacagaatatcatctactgactctgagctttctaccgacaagccaatggctaacatcaaatccacccttagaaagttagatttggttggttttgtcgtttttgctgccttcgctacaatgattagtttggccttagagtggggtgggtccacttacacttggagatcatctgtaatcatcggcttgttttgtggaggtggcttcgcattgattgctttcgttctatgggaaaggcacgtaggagatgcagtcgcaatgattccaggttctgtggcaggaaaaagacaggtgtggtgctcttgcttgtttatgggctttttctcaggttcacttttagtattctcttactacctgcctatctacttccaggcagtcaaagatgtttctccaactttgagtggagtgtatatgctgcctggtatcctaggtcaagtcatcatggcaatggtttccggtttcgcaatcggtaaaaccggctactacttaccatgggctttggggagtgccgtactagttgcaataggggctggtttagtgtctacatttcaacctcatacatctacagtgaagtgggtaatgtaccaattcatcgcaggctttggaagaggctgcggtatgcaaacacctatcatcgccattcaatcaacattaagtccagaacaaggtgccctagggatatctttggcagtttttggtcaaacttttggaggatcattgtttttggactttgccaacttagtctttggttctggattgaggactggcttatctaagtacgccccaacagttgatacacaggcagtaacagcagctggtgcaacaggttttagagatgtggtttccaaaaacaatcttcctggggttgttaaggcatattcattagcagtcgatcatacattctatctagcagtgggagctacagcttgcacttttgtatttgcatttggaatggggtggagaaagatagctaccaaaaacgatactagagcagtccctgagactgatgcataa

SEQ ID NO: 3 - DNA, 2337 bp, Artificial Sequence
  Fungi
atgagtgaaccattaccaccaaaggaaggggaaccaagaccacaaaaagaggaatctcaaaatgacacactggaagctaccgaatcaaaatcccagcacataaccggccttaaacttgggttggtagtagcatctgttaccttcgttgctttccttatgttactagatatgtccatcattgtaacagctattcctcacattacatcagaattccattcattgaatgatgttggctggtacggtagtgcttatcttttagctaattgtgccctgcaaccattagctggcaaattgtatactttgctggggttaaagtacacctttttcgcctttttgtgtatcttcgaattaggatctgtcctatgtggtgctgctcgtagttctactatgttaatcgtcgggcgtgccgtggccggtatgggcggatccggtttggtcaatggtgctctgacaatcttgtcaactgcagcccctaaacataagcagcctgttctaataggtgttatgatgggtctatcccaaattgctattgtgtgtggcccattgcttggcggtgcttttactcaacatgccacatggagatggtgtttctacatcaatctgccaattggtgccgttgccgcttttctactattggtgattacaattccagacagaatatcatctactgactctgagctttctaccgacaagccaatggctaacatcaaatccacccttagaaagttagatttggttggttttgtcgtttttgctgccttcgctacaatgattagtttggccttagagtggggtgggtccacttacacttggagatcatctgtaatcatcggcttgttttgtggaggtggcttcgcattgattgctttcgttctatgggaaaggcacgtaggagatgcagtcgcaatgattccaggttctgtggcaggaaaaagacaggtgtggtgctcttgcttgtttatgggctttttctcaggttcacttttagtattctcttactacctgcctatctacttccaggcagtcaaagatgtttctccaactttgagtggagtgtatatgctgcctggtatcctaggtcaagtcatcatggcaatggtttccggtttcgcaatcggtaaaaccggctactacttaccatgggctttggggagtgccgtactagttgcaataggggctggtttagtgtctacatttcaacctcatacatctacagtgaagtgggtaatgtaccaattcatcgcaggctttggaagaggctgcggtatgcaaacacctatcatcgccattcaatcaacattaagtccagaacaaggtgccctagggatatctttggcagtttttggtcaaacttttggaggatcattgtttttggactttgccaacttagtctttggttctggattgaggactggcttatctaagtacgccccaacagttgatacacaggcagtaacagcagctggtgcaacaggttttagagatgtggtttccaaaaacaatcttcctggggttgttaaggcatattcattagcagtcgatcatacattctatctagcagtgggagctacagcttgcacttttgtatttgcatttggaatggggtggagaaagatagctaccaaaaacgatactagagcagtccctgagactgatgcaatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcctag

SEQ ID NO: 4 - DNA, 1629 bp, Artificial Sequence
  Fungi
atgacatcccaccacggtgaaacagagaagccacagagcaacacggctcaaatgcagataaatcatgtcactggcctcaggctaggcctggttgtggtttcagtcactctggtggcgtttctgatgctcttggatatgtccatcattgtcacggcgattcctcacattaccgcccagtttcattccctgggcgatgtcggatggtacggaagtgcgtatcttctatcaagctgtgccctccaacccttggcaggcaaactatacactctgttgaccctgaaatacaccttcctcgcttttctcgggttgtttgagattggatcggttctttgcggcactgctcgttcgtcaaccatgttgattgtagggcgagcagtggccggaatgggagggtcggggctcaccaatggcgcaatcaccattctgtcggcggcagctccaaagcaacagcaaccgctcttgattgggatcatgatgggcctaagccaaatcgccattgtatgtggaccgttgcttgggggtgctttcacgcagcacgcaagttggcggtggtgtttttacatcaaccttcccattggggcgtttgccacatttctccttctcgtcatccagatccccaacagattgccatccacgtcggattcaaccacagacggcacaaaccccaagagaagaggggctcgggacgtcttgacccaactggatttccttggattcgtgctcttcgccggttttgcgatcatgatatctcttgctttggagtggggtgggtctgattatgcgtggaatagttccgtgatcatcggcttgttctgtgcggcgggcgtgtcgctggtgctgttcggatgctgggaacggcatgtcggcggtgcagtggccatgattcccatttccgtggccagtcgtcgccaagtctggtgctcctgcttcttcctcggctttttttccggggccctacttattttctcctactacctgcctatctacttccaggcggtcaagaatgtttctcccaccatgagtggagtgtatatgctgccgggcattggtggacagatcgtcatggcgattgtgacgggtgcaatcatcggtaaaacaggctattacgttccgtgggcgctcgcaagcgggatccttgtgtccatatccgccggactggtatcgaccttccagccggaaacctcgattgcagcatgggtcatgtatcagttcctgggaggcgtgggccgaggatgcggaatgcaaacccctgtcgtcgccattcaaaatgcgctgcctccacaaacgagccccatcggcatttcgctagccatgttcggccagacattcggtggctcgctttttctcaccctgaccgaattggtttttagcaatggtttggactctggtctgcgccaatatgcgccaaccctcaatgcacaggaggtaacagccgcaggggccaccggcttccgccaagtggtccccgctcctctcatctctcgggtcctcttagcatacagtaaaggcgtggaccatgcattctacgttgcggtcggtgcgtctggagctaccttcatcttcgcctggggtatgggccggcttgcctggagaggctggcggatgcaggagaaaggacggagcgaatga

SEQ ID NO: 5 - DNA, 1632 bp, Artificial Sequence
  Fungi
atggcttcccaccagtctgagaaagagaagccacagagctgcaccactgaagtccaggtcagccatgtcaccggcctaaagctaggactggtagtaacctcggtgaccctggtggtgtttttgatgttgttggacatgtctatcattgtgacagctatcccccatatcactgctcagtttcattcccttggggatgttggatggtatggaagtgcgtatcttttgtcgagctgtgctctacagcccttggcagggaagctctacactctcttgacattgaagtataccttcctagcattcctcggggtgtttgaagtcggatcagctctctgtggcgccgcgcgttgttcaactatgttgatcgtggggcgcgcagtggctggcatgggaggatcggggctcaccaatggagccatcaccatcctcgcctctgcagctccaaaacaacagcaaccactgttaatcgggatcatgatgggtctgagccaaattgccattgtctgtggaccattgctgggaggtgcttttacccaacacgcaagttggcggtggtgcttctatatcaatcttcctgtcggagcgctggccgccatcctccttctcgccatccacattcctaagagtgtgccaacatcggattgcacaatgcctgctcccagagctgttggggtccgggtcatcctgagtcagctcgatctcctggggtttgtgctcttcgccgcctttgccgtgatgatctcacttgcgttagaatggggcgggtccgattatatgtgggatagctccgtaatcatcggcttgttctgtggtgccggcatctcgctggtggtgtttgggttctgggaacgctacgtaggcaactcgatggcgatgattcctttctcggtggccagtcgtcgacaagtctggtgctcgtgtcttttcttgggctttttctccggggccttgctcaccttctcttactaccttcctatctacttccaggccgtgaaggacgtctctcccaccatgagtggggtgtatatgcttccaggcatagggggacaaattgtgatggcgatcgtctccggcgcaatcatcggcaaaacggggtattacattccctgggcgcttgccagtgggatcatcgtatctatctccgcaggcctggtatcgaccttccagccgcatacctcaatcgcagcgtgggtgatgtatcaattcatggggggctttggtcgaggatgtggaatgcagacccccatcattgccattcaacatgccctgccaccacaaatgagtgcgctcggtatctcgctggccatgttcggccagaccttcggcggctccctcttcctcaccttggccaagctcgtcttcagcgccggccttgacgccggcttacgcgagtatgcgcccgccgtcagcgcagaggcggtgacggccgcgggcgccacgggcttccgcgatgtcgtccccgcaaatctcctttctcaggttctcctggcatactgtaaagggatagaccatacattctaccttgcggtcggggcatcgggagccacttttttgtttgcgtggggaatgggtcaggtcggtttgatttggtggggggaggaaaggacggggttcggccgcgatgaacgagtctag

SEQ ID NO: 6 - DNA, 31 bp, Artificial Sequence
  /note=“Primer (combined DNA/RNA)” /note=“Combined DNA/RNA”
agcgatacguaaaaatgagtgaaccattacc

SEQ ID NO: 7 - DNA, 25 bp, Artificial Sequence
  Primer (combined DNA/RNA)
cacgcgauttatgcatcagtctcag

SEQ ID NO: 8 - DNA, 33 bp, Artificial Sequence
  Primer (combined DNA/RNA)
acgtatcgcugtgagtcgtattacggatccttg

SEQ ID NO: 9 - DNA, 30 bp, Artificial Sequence
  Primer (combined DNA/RNA)
cgtgcgaugccgcttgttttatatttgttg

SEQ ID NO: 10 - DNA, 30 bp, Artificial Sequence
  Primer (combined DNA/RNA)
atggcctccuccgaggacgtcatcaaggag

SEQ ID NO: 11 - DNA, 30 bp, Artificial Sequence
  Primer (combined DNA/RNA)
cacgcgauctaggcgccggtggagtggcgg

SEQ ID NO: 12 - DNA, 28 bp, Artificial Sequence
  Primer (combined DNA/RNA)
aggaggccautgcatcagtctcagggac

SEQ ID NO: 13 - DNA, 29 bp, Artificial Sequence
  Primer (combined DNA/RNA)
agcgatacguaaaaatggcctcctccgag

SEQ ID NO: 14 - DNA, 21 bp, Artificial Sequence
  Primer
cttgagtaactctttcctgta

SEQ ID NO: 15 - DNA, 59 bp, Artificial Sequence
  Primer
taagttttcgtatccgctcgttcgaaagactttagacaaaacttcgtacgctgcaggtc

SEQ ID NO: 16 - DNA, 62 bp, Artificial Sequence
  Primer
tcttggtaagtttcttttcttaaccaaattcaaaattctactagtggatctgatatcaccta

SEQ ID NO: 17 - protein, 557 aa, Artificial Sequence
  Fungi
MLCEMSEPLPPKEGEPRPQKEESQNDTLEATESKSQHITGLKLGLVVASVTFVAFLMLLDMSIIVTAIPHITSEFHSLNDVGWYGSAYLLANCALQPLAGKLYTLLGLKYTFFAFLCIFELGSVLCGAARSSTMLIVGRAVAGMGGSGLVNGALTILSTAAPKHKQPVLIGVMMGLSQIAIVCGPLLGGAFTQHATWRWCFYINLPIGAVAAFLLLVITIPDRISSTDSELSTDKPMANIKSTLRKLDLVGFVVFAAFATMISLALEWGGSTYTWRSSVIIGLFCGGGFALIAFVLWERHVGDAVAMIPGSVAGKRQVWCSCLFMGFFSGSLLVFSYYLPIYFQAVKDVSPTLSGVYMLPGILGQVIMAMVSGFAIGKTGYYLPWALGSAVLVAIGAGLVSTFQPHTSTVKWVMYQFIAGFGRGCGMQTPIIAIQSTLSPEQGALGISLAVFGQTFGGSLFLDFANLVFGSGLRTGLSKYAPTVDTQAVTAAGATGFRDVVSKNNLPGVVKAYSLAVDHTFYLAVGATACTFVFAFGMGWRKIATKNDTRAVPETDA

SEQ ID NO: 18 - protein, 542 aa, Artificial Sequence
  Fungi
MTSHHGETEKPQSNTAQMQINHVTGLRLGLVVVSVTLVAFLMLLDMSIIVTAIPHITAQFHSLGDVGWYGSAYLLSSCALQPLAGKLYTLLTLKYTFLAFLGLFEIGSVLCGTARSSTMLIVGRAVAGMGGSGLTNGAITILSAAAPKQQQPLLIGIMMGLSQIAIVCGPLLGGAFTQHASWRWCFYINLPIGAFATFLLLVIQIPNRLPSTSDSTTDGTNPKRRGARDVLTQLDFLGFVLFAGFAIMISLALEWGGSDYAWNSSVIIGLFCAAGVSLVLFGCWERHVGGAVAMIPISVASRRQVWCSCFFLGFFSGALLIFSYYLPIYFQAVKNVSPTMSGVYMLPGIGGQIVMAIVTGAIIGKTGYYVPWALASGILVSISAGLVSTFQPETSIAAWVMYQFLGGVGRGCGMQTPVVAIQNALPPQTSPIGISLAMFGQTFGGSLFLTLTELVFSNGLDSGLRQYAPTLNAQEVTAAGATGFRQVVPAPLISRVLLAYSKGVDHAFYVAVGASGATFIFAWGMGRLAWRGWRMQEKGRSE

SEQ ID NO: 19 - protein, 543 aa, Artificial Sequence
  Fungi
MASHQSEKEKPQSCTTEVQVSHVTGLKLGLVVTSVTLVVFLMLLDMSIIVTAIPHITAQFHSLGDVGWYGSAYLLSSCALQPLAGKLYTLLTLKYTFLAFLGVFEVGSALCGAARCSTMLIVGRAVAGMGGSGLTNGAITILASAAPKQQQPLLIGIMMGLSQIAIVCGPLLGGAFTQHASWRWCFYINLPVGALAAILLLAIHIPKSVPTSDCTMPAPRAVGVRVILSQLDLLGFVLFAAFAVMISLALEWGGSDYMWDSSVIIGLFCGAGISLVVFGFWERYVGNSMAMIPFSVASRRQVWCSCLFLGFFSGALLTFSYYLPIYFQAVKDVSPTMSGVYMLPGIGGQIVMAIVSGAIIGKTGYYIPWALASGIIVSISAGLVSTFQPHTSIAAWVMYQFMGGFGRGCGMQTPIIAIQHALPPQMSALGISLAMFGQTFGGSLFLTLAKLVFSAGLDAGLREYAPAVSAEAVTAAGATGFRDVVPANLLSQVLLAYCKGIDHTFYLAVGASGATFLFAWGMGQVGLIWWGEERTGFGRDERV

SEQ ID NO: 20 - protein, 778 aa, Artificial Sequence
  Fungi
MSEPLPPKEGEPRPQKEESQNDTLEATESKSQHITGLKLGLVVASVTFVAFLMLLDMSIIVTAIPHITSEFHSLNDVGWYGSAYLLANCALQPLAGKLYTLLGLKYTFFAFLCIFELGSVLCGAARSSTMLIVGRAVAGMGGSGLVNGALTILSTAAPKHKQPVLIGVMMGLSQIAIVCGPLLGGAFTQHATWRWCFYINLPIGAVAAFLLLVITIPDRISSTDSELSTDKPMANIKSTLRKLDLVGFVVFAAFATMISLALEWGGSTYTWRSSVIIGLFCGGGFALIAFVLWERHVGDAVAMIPGSVAGKRQVWCSCLFMGFFSGSLLVFSYYLPIYFQAVKDVSPTLSGVYMLPGILGQVIMAMVSGFAIGKTGYYLPWALGSAVLVAIGAGLVSTFQPHTSTVKWVMYQFIAGFGRGCGMQTPIIAIQSTLSPEQGALGISLAVFGQTFGGSLFLDFANLVFGSGLRTGLSKYAPTVDTQAVTAAGATGFRDVVSKNNLPGVVKAYSLAVDHTFYLAVGATACTFVFAFGMGWRKIATKNDTRAVPETDAMASSEDVIKEFMRFKVRMEGSVNGHEFEIEGEGEGRPYEGTQTAKLKVTKGGPLPFAWDILSPQFQYGSKAYVKHPADIPDYLKLSFPEGFKWERVMNFEDGGVVTVTQDSSLQDGEFIYKVKLRGTNFPSDGPVMQKKTMGWEASTERMYPEDGALKGEIKMRLKLKDGGHYDAEVKTTYMAKKPVQLPGAYKTDIKLDITSHNEDYTIVEQYERAEGRHSTGA

(19) United States
(12) Patent Application Publication
(10) Pub. No.: US 2017/0088589 A1
(43) Pub. Date: Mar. 30, 2017

(54) STATIN RESISTANCE AND EXPORT

(71) Applicant: DANMARKS TEKNISKE UNIVERSITET, Kgs. Lyngby (DK)

(72) Inventors: Rasmus John Normand Frandsen, Allerød (DK); Ane Ley, Kgs. Lyngby (DK); Michael Naesby, Huningue (FR)

(21) Appl. No.: 15/305,787

(22) PCT Filed: Apr. 16, 2015
(86) PCT No.: PCT/DK2015/050098

§ 371 Date: Oct. 21, 2016

(30) Foreign Application Priority Data
Apr. 23, 2014 (EP) 14165696.7

(51) Int. Cl. C07K 14/395; C12P 17/06

(52) U.S. Cl. CPC C07K 14/395; C12P 17/06

(57) ABSTRACT

The present invention relates e.g. to methods of producing statins in transgenic, non-filamentous microorganisms such as Saccharomyces cerevisiae. In addition, the present invention relates to the transgenic, non-filamentous microorganisms as such as well as various uses of transmembrane statin efflux pump(s) originating from various filamentous fungi. Moreover, the present invention relates to the transferring the compactin, lovastatin or monacolin K gene cluster originating from non-filamentous fungi into easily fermentable microorganisms, followed by expression or overexpression of the efflux pump encoding genes in said microorganisms in order to increase the microorganisms resistance to statins which in turn allows for production of elevated concentrations of natural statins compared to statin-producing methods known in the art.

### TECHNICAL FIELD OF THE INVENTION

The present invention relates e.g. to methods of producing statins in transgenic, non-filamentous microorganisms such as Saccharomyces cerevisiae. Further, the present invention relates to the transgenic, non-filamentous microorganisms as such as well as various uses of transmembrane statin efflux pump(s) originating from various filamentous fungi.

### BACKGROUND OF THE INVENTION

Statins are important inhibitors of 3-hydroxy-3-methylglutaryl coenzyme A reductase (HMGR), the regulatory and rate-limiting enzyme in the mevalonate pathway, which leads to the production of sterols, such as cholesterol in human, and ergosterol in fungi.

The blood cholesterol level in mammals is a result of de novo synthesis and dietary intake. Elevated levels of blood cholesterol often lead to atherosclerosis, i.e. deposits of LDL particles on the inside of the arterial walls, leading to various cardiovascular diseases. Treatment of elevated cholesterol levels is typically a combination of dietary changes and medical treatment with statins to control de novo synthesis.

With their effective cholesterol-lowering ability, statins have been widely used as hypercholesterolemia drugs to prevent and treat cardiovascular diseases and has become one of the best-selling pharmaceuticals in the past decade.

Typically, statins are divided into three classes based on their mode of synthesis: natural, semi-natural and synthetic. Natural statins, such as lovastatin/monacolin K and compactin, are synthesised via the polyketide biosynthetic pathway by filamentous fungi including Aspergillus terreus, Monascus purpureus and Penicillium citrinum. The natural statin compounds are utilized by the fungi to inhibit the growth of eukaryotic competitors that inhabits the same ecosystems/niche. Semi-natural statins are natural statins, which post-purification has been modified through synthetic chemistry or via a biotransformation. The synthetic statins differ significantly in structure from that of the natural and semi-natural statins and are produced by chemical synthesis.

More specifically, the two known natural statins, compactin and lovastatin, are produced as secondary metabolites by filamentous fungi; compactin is produced by Penicillium species, e.g. P. solitum and P. brevicompactum, whereas lovastatin is produced by Aspergillus and Monascus species, e.g. A. terreus, M. purpureus and M. pilosus.

In general, bioactive secondary metabolites often provide a selective advantage to the producing microorganism in their natural environment. However, said metabolites can also be toxic to the producing microorganisms if they themselves contain the target site of the compound. Therefore, the secondary metabolite biosynthesis gene clusters, in addition to the biosynthetic enzymes, often also contain genes encoding a secretion system(s), which in addition can provide a resistance mechanism to prevent self-intoxication.

This is also the case in relation to both the compactin and the lovastatin biosynthesis gene clusters, where putative efflux pump genes have been identified, namely the mlcE gene from the compactin biosynthetic gene cluster, lovI gene (also referred to in the art as: ORF10 or lovH—thus, for the purpose of simplicity the gene is hereinafter referred to as lovI/H) from the lovastatin gene cluster and mokI from the monacolin K cluster.

Well-known methods of producing natural statins, as well as their semi-synthetic derivatives, are mainly based on fermentation processes with strains of naturally statin-producing filamentous fungi.

Commercial production of natural and semi-natural statins is based on liquid fermentation of the relevant fungal species followed by purification and subsequent modification for the semi-natural statins. However, it is also well known that filamentous fungi are difficult to culture efficiently in fermenters, inter alia due to their unique physiology and morphology.

Hence, in order to overcome these problems and to increase the yields, several statin-manufacturing companies have switched to solid state fermentation, a challenging approach that is prone to contamination and involves a relatively high risk for the formation of undesirable side products. This is also well known in the art.

Also, it is well-known in the art that there is a common problem while fermenting statins in fungi as the final products are—besides being cholesterol lowering agents—also active antifungals and thereby limit the productivity in fungal hosts. A possible solution to this problem could be to transfer the metabolic pathway to easily fermentable unicellular microorganisms, such as yeast. However, this solution is not easily achievable inter alia since yeast does not naturally produce any polyketides, which is one of the reasons why the relevant genes encoding the biosynthetic machinery for the formation of statins have to be functionally expressed simultaneously at a balanced level. Additional challenges for producing statins in yeast include a limited availably of the necessary substrates (acetyl-CoA and malonyl-CoA) and co-factors (NADPH). Further challenges includes problem with self-intoxication as yeast only has a basal level of statin-resistance (Riccardo & Kielland-Brandt, 2011).

Thus, in an effort to provide an alternative mode of biosynthesis, researcher has for the last decade been working on transferring the statin biosynthetic pathway from the traditionally used filamentous fungi into easily fermentable microorganisms such as Saccharomyces cerevisiae.

Xu W. et al. (2013), discloses inter alia the expression in yeast of the genes responsible for the biosynthesis of the lovastatin intermediate, monacolin J acid. However, the Xu W. et al. -article did not contain any disclosure of the expression of the statin efflux pump genes mlcE, mokI and/or lovI/H in e.g. Saccharomyces cerevisiae, but merely discloses the expression of genes responsible for the biosynthesis of monacolin J acid which is an intermediate capable of being converted into the commercially attractive agent, simvastatin acid, in a single enzymatic step.

Abe et al. (2002) discloses inter alia that mlcE is a putative efflux pump which may be involved in conferring resistance to compactin as well as in metabolite secretion in the naturally producing microorganism (Penicillium citrinum). However, the Abe et al.-article contains no disclosure or suggestions of transferring and/or expressing the mlcE, mokI or lovI/H genes in yeast, let alone in Saccharomyces cerevisiae.

An article by Hirata D., & Yano K. (1994), discloses inter alia that the pdr5 gene encodes an efflux pump in Saccharomyces cerevisiae.

Likewise, in an article by Riccardo L., & Kielland-Brandt M C. (2011) is disclosed that pdr5 gene encodes a pump that has shown to confer basic-level of statin-resistance in Saccharomyces cerevisiae. Also disclosed in said article is the susceptibility to lovastatin of Saccharomyces cerevisiae strains deleted for PDR genes, i.e. genes encoding for drug resistance pumps responsible for exporting hydrophobic and amphiphilic drugs, such as lovastatin.

WO09133089 A1 disclosed inter alia a process for increasing the compactin, pravastatin, lovastatin and/or simvastatin productivity by a fermentation process carried out with host organisms that are genetically engineered to have increased resistance to said statins. More specifically, a process is provided which makes use of microorganisms (preferably Penicillium chrysogenum) in which genes encoding proteins which mediate statin resistance are overexpressed. Also disclosed in WO09133089 A1 is the compactin biosynthetic gene cluster of Penicillium citrinum (i.e. mlcA, mlcB, mlcC, mlcD, mlcE, mlcF, mlcH, mlcG, mlcR) as well as the lovastatin biosynthetic gene cluster of Aspergillus terreus (i.e. ORF1, ORF2, lovA, ORF5, lovC, lovD, ORF8, lovE, ORF10, lovF, ORF12, ORF13, ORF14, ORF15, ORF16, ORF18). However, WO09133089 A1 contains no disclosure of neither the functions of said genes nor the transferring and expression of the statin efflux pump encoding lovI/H, mokI or mlcE genes in Saccharomyces cerevisiae.

WO0129073A1 disclosed to the use of so-called MFS-transporters (named PUMPI and PUMP2) and their ability to confer resistance to otherwise toxic levels of lovastatin when expressed in Saccharomyces cerevisiae. However, as there is no disclosure or suggestion in WO0129073A1 of transferring and expressing the statin efflux pump encoding lovI/H, mokI or mlcE genes in Saccharomyces cerevisiae.

WO0037629 disclosed inter alia a method of increasing the production of lovastatin in a lovastatin-producing or a non-lovastatin-producing microorganism. ORF10 (lovI/H) is disclosed in WO0037629 as a gene relevant for the transportation of metabolites. There is no disclosure in WO0037629 of lovI/H, mokI or mlcE, let alone of the transfer and expression of said genes in a Saccharomyces cerevisiae host e.g. for improving the statin resistance in said host.

WO2009/077523 disclosed inter alia a method for the fermentative production of e.g. compactin (mevastatin) and lovastatin where said method comprises culturing a mutant host capable of producing e.g. lovastatin wherein the esterase activity in said mutant host is more than 25% below the activity of said esterase in the parent host. However, there is no disclosure in WO2009/077523 of e.g. the mlcE, mokI and/or lovI/H genes encoding statin specific efflux pumps let alone of transferring and expression of said genes in Saccharomyces cerevisiae.

WO2007147827 discloses inter alia Saccharomyces cerevisiae containing a compactin biosynthesis gene and a gene for conversion of compactin into pravastatin. The mlcE gene is disclosed as being one of these compactin biosynthesis genes. There is no disclosure or suggestion in WO2007147827 that e.g. mlcE, mokI and/or lovI/H is capable of encoding statin specific efflux pumps in Saccharomyces cerevisiae, let alone of the transferring and expression of said genes in Saccharomyces cerevisiae.

WO2010034686 discloses inter alia method for the fermentative production of e.g. compactin (mevastatin) and lovastatin where the method involves culturing a host, e.g. Saccharomyces cerevisiae, e.g. by use of the lovE transcription regulator gene. There is, however, no disclosure or suggestion in WO2010034686 of e.g. the mlcE, mokI and/or lovI/H genes is/are capable of encoding statin specific efflux pumps in e.g. Saccharomyces cerevisiae, let alone of a transfer and expression of said genes in Saccharomyces cerevisiae.

WO10069914 discloses a method for the fermentative production of e.g. compactin (mevastatin) and lovastatin where the method involves culturing a host, e.g. Saccharomyces cerevisiae, e.g. by use of specifically defined transcription regulator genes.

WO10069914 also discloses that mlcE encoding an efflux pump in Penicillinum citrinum. However, WO10069914 contains no disclosure or suggestions that said efflux pump gene can be expressed in e.g. Saccharomyces cerevisiae.

As is apparent from the above-outlined prior art documents, the three genes mlcE, mokI and lovI/H have not previously been characterized in depth, let alone when transferred into other host organisms. The article by Hutchinson et al. (2000), discloses the function of the lovI gene and states inter alia that heterologous expression of the putative lovastatin efflux pump gene lovI in Aspergillus nidulans did not result in increased resistance to lovastatin in said host organism (no experimental data is provided in the article). Moreover, said article contains no disclosure of e.g. expressing the mlcE, mokI and/or lovI/H genes in yeast.

It is well known that statins are toxic for the statin-producing host cells, e.g. due to the inhibition of ergosterol biosynthesis (fungal equivalent of cholesterol). It is therefore crucial to establish a nondestructive resistance mechanism in a given host cell (said host cell is also commonly referred to as a “cell factory”) in order to establish a commercially profitable production of statins.

In order to avoid the undesirable effects of self-intoxication in the host cell several approaches has previously been utilized the most common being: 1) overexpression of the HMGR encoding gene and/or 2) development of a statin-insensitive HMGR.

The present invention relates to a novel approach for avoiding the undesirable effects of self-intoxication in easily fermentable host microorganisms by introduction of a transmembrane statin efflux pump in said microorganism for removing the toxic statins from said host.

This novel approach has the additional advantage that it also ensures the export out of microorganism of any produced statins, which inter alia eases the subsequent purification steps.

Hitherto, however, it has not been clear whether the putative efflux pumps from the statin biosynthetic gene clusters have the potential to export statins out of the statin-producing microorganisms. Thus, the inventors of the present application surprisingly found that introduction of genes encoding transmembrane statin efflux pumps, such as the mlcE, mokI and/or lovI/H gene(s) into statin sensitive yeast hosts was indeed feasible and additionally found that said introduction turned out to increase the yeast's resistance to statins present in the relevant growth media.

Hence, to summarize, there is a need for improvement in the art of the productivity of fungal fermentations due the anti-fungal properties of statins. Drawbacks of the state of the art processes of producing statins—which are overcome by the present invention—involve inter alia:

(i) the fact that filamentous fungi, traditionally used for statin-production, are difficult to culture efficiently in fermenters, inter alia due to their unique physiology and morphology.

(ii) the potential negative effects of the traditionally used method of overexpression of HMGR (in order to reduce statin self-intoxication) might have on the central metabolism of the host microorganism

(iii) the potentially deleterious effects of the statin self-intoxication of the statin-producing host microorganisms

(iv) the contamination problems and the risk of the formation of undesirable side products associated with the traditionally used “solid state fermentation” methods of producing statins. Moreover, it is well known in the art that collecting and/or purifying the produced statin in the traditionally used “solid state fermentation” is both laborious and cost-ineffective.

### SUMMARY OF THE INVENTION

The object of the present invention is inter alia to provide a method to solve some of the problems encountered in prior art processes of producing statins. Preferably, a process is provided which makes use of easily fermentable microorganisms, such as Saccharomyces cerevisiae, in which genes encoding statin efflux pumps are overexpressed.

Based on the hypothesis that the proteins MlcE, LovI/H and MokI—from the compactin, lovastatin and monacolin K gene clusters respectively—are in fact transmembrane statin specific efflux pumps the inventors of the present application have successfully expressed and overexpressed e.g. the mlcE gene in different Saccharomyces cerevisiae strains and tested the responses of said strains to increasing statin levels.

Thus, the present invention relates to the transferring of the compactin, lovastatin or monacolin K gene cluster into easily fermentable microorganisms, such as Saccharomyces cerevisiae, followed by overexpression of the efflux pump encoding mlcE, mokI and/or lovI/H genes in said microorganisms.

This expression or overexpression turned out to increase resistance to statins in easily fermentable microorganisms such as, Saccharomyces cerevisiae, Pichia pastoris and Schizosaccharomyces pomp.

Moreover, the present invention relates to the use of the transmembrane statin efflux pumps, such as MlcE, for increasing the resistance in transgenic microorganisms to the potentially deleterious effects of exogenous added statins on said microorganisms in connection with production of said statins in the organism.

This reduction of statin self-intoxication in the producing host microorganism allow for the production of elevated concentrations of natural statins compared to statin-producing methods known in the art. Moreover, overexpression in said hosts of the genes encoding the transmembrane statin efflux pumps, i.e. the mlcE, lovI/H and mokI genes, eliminates the potentially adverse effects of overexpression the genes encoding the HMGR enzyme, i.e. one of the methods traditionally used in the art to produce statins in microorganisms. Moreover, expression of the mlcE, lovI/H and mokI genes increased export of statins from the cytoplasma to the growth medium, easing purification of said statins.

### DEFINITIONS

Prior to discussing the present invention in further details, the following terms will first be defined. In the context of the present application, the following terms have the following meanings. The below-outlined terms are listed in alphabetical order:

Activated lovastatin: Lovastatin, as well as other statins comprise of a lactone ring unit, which can be present in an open or closed form, depending on the pH. Statins are biologically active (i.e. are able to inhibit HMGR) only when their lactone ring is in an open confirmation (dihydroxy open-acid form). Activated lovastatin is lovastatin with an open lactone ring.

ARX3 strain: Saccharomyces cerevisiae strain originating from CEN.PK 113-11C strain with the efflux pump encoding gene mlcE from the compactin biosynthetic gene cluster integrated into the genome using method described by Mikkelsen et al., 2012.

Codon optimization: A codon is a DNA entity composed of three nucleotides that is being translated into a specific amino acid residue in a polypeptide chain. The Genetic code is degenerated, meaning that many amino acids can be encoded by more than one codon. Different organisms show preferences for particular codons that encode specific amino acids. Codon optimization is a method for optimizing gene sequences in a way that the amino acid residues of the polypeptide chain are encoded by the codons preferred by the organism, in which we would like to express the gene.

Constitutive promoter (e.g. TEF1): Promoter that is active under all conditions in the cell. Gene expressed under a constitutive promoter is being continuously transcribed in the cell.

Crystal violet efflux pump (Sge1): Sge1 protein from Saccharomyces cerevisiae is a member of the drug-resistance protein family, and is capable of conferring resistance in yeast to crystal violet and other toxic substances.

C-terminal mRFP fusion: In order to determine subcellular localization of proteins, the proteins can be tagged with a reporter protein, e.g. red fluorescent protein (RFP) at their C terminus or N terminus. Because of the ability of RFP to emit light when illuminated with light of a specific wavelength, the fused proteins can be tracked in cells using fluorescence microscopy.

“Drug:H+ antiporter 2 family”: Drug:H+ antiporter 2 family is a family of multidrug resistance transport proteins from the major facilitator superfamily (MFS). Proteins in this family are membrane-bound enzymes containing 14 transmembrane spanning domains. They catalyze a reaction in which hydrogen protons and drugs are pumped in opposite direction across a membrane.

HC-toxin efflux pump (ToxA): ToxA protein form Cochliobolus carbonum is an HC-toxin efflux pump which contributes to self-protection against HC-toxin and/or secretion of HC-toxin into the extracellular environment.

HMGR: 3-hydroxy-3-methylglutaryl coenzyme A reductase (HMGR) is the rate-limiting enzyme in the mevalonate pathway, which leads to the production of sterols, such as cholesterol in human, and ergosterol in fungi. The enzyme is inhibited by products from the mevalonate pathways via a negative feedback loop.

Major facilitator superfamily (MFS): MFS is a family of membrane transport proteins that facilitate movement of small molecules across cell membranes in response to chemiosmotic ion gradients.

Mevastatin: also referred to in the art as compactin (and ML-236B) is a hypolipidemic agent that belongs to the statins class.

MlcE topology: Topology describes the orientation of regular secondary structures, such as alpha-helices and beta strands in a protein structure and in relation to cell membranes. MlcE topology refers to the topology of the efflux pump MlcE.

mRFP: monomeric red fluorescent protein is a reporter protein used in fluorescence microscopy for subcellular localization of proteins to which mRFP is fused.

OD₆₀₀: Optical density (also called absorbance) is a measure of concentration of cells in a suspension. It is determined in a spectrophotometer at a wavelength of 600 nm.

Overexpression: describes the various methods by which a gene or a protein can be modified in order to increase the concentration of active enzyme, including inter alia (i) introduction of additional gene copies encoding host or heterologous proteins; (ii) overexpression of host proteins from a strong promoter; (iii) modifying the transcriptional regulation of the genes encoding enzymes mediating statin resistance; (iv) modifying the mRNA to increase the rate of translation initiation; (v) mutation of critical amino acids leading to proteins with improved kinetic properties; (vi) mutations causing an increased half-life of the enzyme; (vii) modifying the mRNA molecule in such a way that the mRNA half-life is increased; Other methods which are well-known in the art may be envisaged.

pdr5 deletion strain (Pleotropic Drug Resistance gene): the pdr5 gene encodes a pump that has shown to confer a basic level of statin-resistance in Saccharomyces cerevisiae. The pdr5 deletion strain (herein denoted as: pdr5Δ) does not contain said pump.

Plate dilution assay (spot assay): This assay allows testing of the toxic effects of the compounds added to a solid growth medium. It is based on culturing a dilution series of a microorganism on said plates, following the growth of a microorganism and observing at which dilution the microorganism is unable to grow. The growth of individual microorganisms as a function of time is recorded by photography of the plates.

Penicillium citrinum: the compactin-producing filamentous fungi (also referred to in the art as Penicillium solitum)

Recombinant host strains: refers to host strains in which genetic material from one or multiple sources have been brought together, creating sequences that would not otherwise be found in biological organisms.

RFP-tagged MlcE: To investigate subcellular localization of efflux pump MlcE, the relevant protein has been fused with RFP at its C terminus.

Standard Protein BLAST: Basic Local Alignment Search Tool is an algorithm for comparing biological sequence information. Standard Protein BLAST, available at e.g. http://www.ncbi.nlm.nih.gov is commonly used for identifying a query amino acid sequences in protein databases. The search tool is designed to identify local regions of similarity.

Substantially homologous polynucleotide: A polynucleotide with nucleotide sequences that are substantially homologous to a reference sequence is defined as a polynucleotide with a nucleotide sequence with a degree of identity to the specified nucleotide sequence of at least 80%, preferably at least 85%, more preferably at least 90%, still more preferably at least 95%, still more preferably at least 97%, still more preferably at least 98%, most preferably at least 99%

Substantially homologous polypeptide: A polypeptide with amino acid sequences that are substantially homologous to a reference sequence is defined as a polypeptide with an amino acid sequence with a degree of identity to the specified amino acid sequence of at least 80%, preferably at least 85%, more preferably at least 90%, still more preferably at least 95%, still more preferably at least 97%, still more preferably at least 98%, most preferably at least 99%. Substantially homologous polypeptides may for example contain only conservative substitutions of one or more amino acids of the specified amino acid sequences or substitutions, insertions or deletions of non-essential amino acids.

TEF promoter: constitutive promoter that regulates transcription of the ‘Transcription Elongation Factor b’ encoding gene TOPCONS server: TOPCONS server is a tool for consensus prediction of membrane protein topology. It is available online at http://topcons.net/

Wild type strain: Reference Saccharomyces cerevisiae strain, in this study CEN.PK 113-11C (MATa MAL2-8C SUC2 his3Δ ura3-52).

YPD agar plates: YPD is a complete medium for yeast growth composed of yeast extract, peptone and glucose. If YPD is used as a solid medium, agar is added to YPD, and the medium is solidified in plates for cultivation of microorganisms.

Advantages of the Present Invention

The inventors of the present application surprisingly showed that it is possible to use the transmembrane statin efflux pumps MlcE, LovI/H and MokI as a resistance mechanism, i.e. for reducing the potentially deleterious effects of self-intoxication caused by the produced statins in a statin-producing microorganism, e.g. in yeasts such as Saccharomyces cerevisiae. This is a surprising finding in light of the prior art which e.g. suggested that this could not be the case as expression of the mlcE gene in e.g. the filamentous fungus Aspergillus nidulans, which is normally sensitive to statins, did not increase its resistance against the tested compounds (see e.g. the article by Hutchinson et al., 2000).

An additional advantage of the present invention, in addition to providing resistance against both natural and semi-statins, is that the statin efflux pumps also provides an elegant solution for exporting the produced statins into the extracellular medium in statin-producing hosts other than filamentous fungi, e.g. in yeasts such as Saccharomyces cerevisiae.

Furthermore, by expressing e.g. the mlcE gene in easily fermenting hosts such as Saccharomyces cerevisiae there is no longer a need for the traditionally used overexpression of HMGR which in turn eliminates the potential negative effects that HMGR might have on the central metabolism of the host microorganism.

The above-mentioned statin pumps, e.g. MlcE, with their ability of exporting natural and semi-natural statins across the plasma membrane has a great potential for improving a statin-producing yeast cell factory. Not only do said pumps, e.g. MlcE, provide the resistance to a range of statins in yeast; it also ensures the export of the produced statins into the extracellular environment, which can significantly ease the subsequent purification of the produced compounds compared to the traditionally used “solid state fermentation” methods based on naturally producing species of Penicillium, Aspergillus and Monascus.

Hence, the inventors of the present application provided evidence indicating that the polypeptides encoded by the mlcE, LovI/H and MokI genes are transmembrane efflux pumps capable of transporting both natural and semi-natural statins out of the statin-producing host cell.

Moreover, the inventors of the present application showed that MlcE, LovI/H and MokI are statin-specific transporters, with the ability to transport compactin as well as the compactin-related compounds lovastatin, simvastatin and pravastatin, across the plasma membrane. Therefore, in light of the above, overexpression of e.g. mlcE in statin-producing microorganisms, such as Saccharomyces cerevisiae could greatly improve the commercial production of natural and semi-natural statins compared to well-known statin-producing methods.

In general this means that the statin efflux pumps MlcE, LovI/H and MokI provide resistance to both, natural and semi-natural statins, making them great tools for optimizing e.g. yeast cell factories for statin production.

### DETAILED DESCRIPTION OF THE INVENTION

Thus, it is an object of the present invention to provide a statin producing method and statin producing transgenic, non-filamentous microorganisms that solves the above mentioned problems of the prior art.

Thus, one aspect of the invention relates to a method for the production of statin in a transgenic microorganism, wherein the method comprises expression in said microorganism of one or more polynucleotide(s) encoding one or more transmembrane statin efflux pump(s).

In a preferred embodiment, the polynucleotide(s) of said method are chosen from the group consisting of SEQ ID NOs: 1, 2, 3, 4 and/or 5.

In a further preferred embodiment, the polynucleotide(s) of said method is/are chosen from the group consisting nucleotide variants comprising sequences with a degree of identity to any of SEQ ID NOs: 1, 2, 3, 4 and/or 5 of at least: 80%, preferably at least 85%, more preferably at least 90%, still more preferably at least 95%, still more preferably at least 97%, still more preferably at least 98%, most preferably at least 99%. In an even further preferred embodiment, the polynucleotide(s) SEQ ID NOs: 1, 2, 3, 4 and/or 5 of said method is/are overexpressed.

In an even further preferred embodiment, the above-mentioned polynucleotide variant(s) of SEQ ID NOs: 1, 2, 3, 4 and/or 5 is/are overexpressed.

Another aspect of the invention relates to a method for the production of statin in a transgenic microorganism, wherein the method comprises expression in said microorganism of polypeptide(s) chosen from the group consisting of SEQ ID NOs: 17, 18, 19 and/or 20.

In a further preferred embodiment, the polypeptide(s) of said method is/are chosen from the group consisting of variants comprising sequences with a degree of identity to any of SEQ ID NOs: 17, 18, 19 and/or of at least: 80%, preferably at least 85%, more preferably at least 90%, still more preferably at least 95%, still more preferably at least 97%, still more preferably at least 98%, most preferably at least 99%.

In an even further preferred embodiment, the polypeptide(s) of SEQ ID NOs: 17, 18, 19 and/or 20 of said method is/are overexpressed.

In an even further preferred embodiment, the above-mentioned polypeptide variants of SEQ ID NOs: 17, 18, 19 and/or 20 is/are overexpressed.

In an even further preferred embodiment, the transgenic microorganism for use in the production of statins is a non-filamentous fungus selected from the group consisting of Saccharomyces cerevisiae, Pichia pastoris and Schizosaccharomyces pomp.

In an even further preferred embodiment, the present invention relates to statins produced by any of the above methods.

Another aspect of the present invention relates to transgenic microorganism for use in the production of statin, wherein the microorganism comprises one or more polynucleotide(s) encoding one or more transmembrane statin efflux pump(s).

In a preferred embodiment, said transgenic microorganism comprises one or more polynucleotide(s) chosen from the group consisting of SEQ ID NOs: 1, 2, 3, 4 and/or 5.

In a further preferred embodiment, the transgenic microorganism comprises one or more nucleotide variant(s) comprising sequences with a degree of identity to any of SEQ ID NOs: 1, 2, 3, 4 and/or 5 of at least: 80%, preferably at least 85%, more preferably at least 90%, still more preferably at least 95%, still more preferably at least 97%, still more preferably at least 98%, most preferably at least 99%.

In an even further preferred embodiment, the transgenic microorganism comprises one or more polynucleotide(s) according to SEQ ID NOs: 1, 2, 3, 4 and/or 5 which is/are overexpressed.

In an even further preferred embodiment, the transgenic microorganism comprises one or more of the above polynucleotide variants of SEQ ID NOs: 1, 2, 3, 4 and/or 5 which are overexpressed.

In a preferred embodiment, said transgenic microorganism comprises one or more polypeptides chosen from the group consisting of SEQ ID NOs: 17, 18, 19 and/or 20.

In a further preferred embodiment, the transgenic microorganism comprises one or more polypeptide variant(s) comprising sequences with a degree of identity to any of SEQ ID NOs: 17, 18, 19 and/or 20 of at least: 80%, preferably at least 85%, more preferably at least 90%, still more preferably at least 95%, still more preferably at least 97%, still more preferably at least 98%, most preferably at least 99%.

In an even further preferred embodiment, the transgenic microorganism comprises one or more polypeptide(s) according to SEQ ID NOs: 17, 18, 19 and/or 20, which is/are overexpressed.

In an even further preferred embodiment, the transgenic microorganism comprises one or more of the above polypeptide variants of SEQ ID NOs: 17, 18, 19 and/or 20, which is/are overexpressed.

In an even further preferred embodiment, the above transgenic microorganism is a non-filamentous fungus selected from the group consisting of Saccharomyces cerevisiae, Pichia pastoris and Schizosaccharomyces pomp.

Yet another aspect of the present invention relates to use of a transmembrane statin efflux pump in a transgenic non-filamentous statin-producing microorganism, for the production of statins in the microorganism and/or increasing the statin resistance in the microorganism and/or decreasing the statin self-intoxication in the microorganism and/or increasing the export of the produced statins into the extracellular medium.

Also envisaged is the use of the statins obtained in any of the above-outlined methods in the production of a medicament.

A further aspect of the invention concerns a use of a transmembrane statin efflux pump in a microorganism, for the:

(i) bioconversion of statins in the microorganism and/or

(ii) increasing the statin resistance of the microorganism and/or

(iii) increasing the export of statins into the extracellular medium

An even further aspect of the invention concerns a polypeptide selected from the group consisting of SEQ ID NOs: 17, 18, 19 and/or 20 which, when incorporated into a transgenic non-filamentous microorganism, is capable of

(ii) providing statin resistance in the microorganism and/or

(ii) exporting of the produced statins out of the microorganism

Also envisaged is one or more polypeptide variant(s) comprising a sequence with a degree of identity to any of SEQ ID NOs: 17, 18, 19 and/or 20 of at least: 80%, preferably at least 85%, more preferably at least 90%, still more preferably at least 95%, still more preferably at least 97%, still more preferably at least 98%, most preferably at least 99% which, when incorporated into a transgenic non-filamentous microorganism, is capable of

(ii) providing statin resistance in the microorganism and/or

(ii) exporting of the produced statins out of the microorganism

A further aspect of the invention concerns a nucleic acid construct comprising any of the polynucleotide sequence according to SEQ ID NOs: 1, 2, 3, 4 and/or 5 operably linked to one or more control sequences that facilitate production of the polypeptide in an expression host.

An even further aspect of the invention concerns a recombinant expression cassette comprising said construct either maintained in the expression host as a self-replicating plasmid or integrated into the genome.

All patent and non-patent references cited in the present application, are hereby incorporated by reference in their entirety.

### BRIEF DESCRIPTION OF THE FIGURES AND THE SEQ ID NOs

FIG. 1:

Phylogenetic tree of representative members of the two families of MFS drug resistance proteins; family DHA1 with Drug:H+ antiporters consisting of 12 TMS and family DHA2 with Drug:H+ antiporters consisting of 14 TMS are shown.The three putative statin efflux pumps (MlcE, LovI and MkI from compactin, lovastatin and monacolin biosynthetic gene clusters, respectively) are predicted to belong the DHA2 family. Protein sequences were obtained from UniProt Knowledgebase (UniProtKB, http://www.uniprot.org/help/uniprotkb), aligned with multiple sequence alignment tool MAFFT version 7 (Multiple sequence Alignment using Fast Fourier Transform) available at the European Bioinformatics Institute (http://mafft.cbrc.jp/alignment/server/). The tree was generated with ClustalW2 alignment program at EMBL-EBI using Neighbor-Joining clustering method (Setting: distance correction on, exclude gaps on), and viewed with FigTree software, version 1.4 (http://tree.bio.ed.ac.uk/software/figtree).

FIG. 2:

Strain construction and subcellular localization of MlcE-RFP. A) Schematic representation of strain construction. Expression cassettes containing mlcE and its RFP-tagged version were integrated into the genome, under the control of the constitutive promoter TEF1 (strains ARX3 and ARX1, respectively). RFP alone has been expressed from the same locus and promoter, and the resulting strain (strain ARX2) was used as a control for fluorescence microscopy. B) Fluorescence microscopy results. For subcellular localization of the putative efflux pump MlcE strain ARX1, and a control strain ARX2 were incubated overnight in 10 mL of SC medium, shaking (150 rpm) at 30° C. Images obtained by differential interference contrast microscopy (DIC) (left panel) and corresponding fluorescence images (right panel) are shown.

FIG. 3:

Investigation of the potential of MlcE to confer the resistance to statins in yeast. Tenfold dilution series of Saccharomyces cerevisiae WT and ARX3 strain harbouring the putative efflux pump MlcE, starting with an OD600 of 0.02 were prepared from overnight cultures. 4.5 microliters of each dilution were plated on a set of YPD agar plates containing different cytotoxic compounds. The plates were incubated at 30° C. for 2 days, after which the growth of the strains was recorded by photography.

FIG. 4:

Investigation of the potential of MlcE to complement the PDR5 efflux pump in yeast. Fivefold dilution series of Saccharomyces cerevisiae WT, ARX3, AR29 pdr5Δ and pdr5Δ strains, starting with an OD₆₀₀ of 0.2 were prepared from overnight cultures. Four microliters of each dilution were plated on a set of YPD agar plates with increasing concentration (0.74 mM and 1.98 mM) of lovastatin. The plates were incubated at 30° C. for 3 days, after which the growth of the strains was recorded by photography.

SEQ ID NO: 1 represents the nucleotides of mlcE (coding sequence, from the compactin biosynthetic gene cluster (GenBank accession number: AB072893.1))

SEQ IN NO: 2 represents the nucleotides of mlcE (coding sequence, synthetic codon optimized version)

SEQ ID NO: 3 represents the nucleotides of mlcE-mRFP (coding sequence, synthetic codon optimized version of mlcE with mRFP fusion)

SEQ ID NO: 4 represents the nucleotides of IovI/H (coding sequence (GenBank accession number: AF141925.1))

SEQ ID NO: 5 represents the nucleotides of mokI (coding sequence (GenBank accession number: DQ176595.1))

SEQ ID NO: 6 represents the nucleotides of the primer mlcE-F

SEQ ID NO: 7 represents the nucleotides of the primer mlcE-R

SEQ ID NO: 8 represents the nucleotides of the primer TEF1-d

SEQ ID NO: 9 represents the nucleotides of the primer PGK1-s

SEQ ID NO: 10 represents the nucleotides of the primer RFP_F+

SEQ ID NO: 11 represents the nucleotides of the primer RFP_{—l R+}

SEQ ID NO: 12 represents the nucleotides of the primer mlcE-RFP-R

SEQ ID NO: 13 represents the nucleotides of the primer RFP-F

SEQ ID NO: 14 represents the nucleotides of the primer C1_TADH1_F

SEQ ID NO: 15 represents the nucleotides of the primer PDR5-DEL-F

SEQ ID NO: 16 represents the nucleotides of the primer PDR5-DEL-R

SEQ ID NO: 17 represents the amino acids of MlcE (GenBank accession number: BAC20568.1)

SEQ ID NO: 18 represents the amino acids of LovI/H (GenBank accession number: AAD34558.1)

SEQ ID NO: 19 represents the amino acids of MokI (GenBank accession number: ABA02247.1)

SEQ ID NO: 20 represents the amino acids of MlcE-mRFP

The invention will now be described in further details in the following non-limiting examples.

### EXAMPLE 1

### Integration of the mlcE Gene into Saccharomyces cerevisiae

General Setup

The mlcE gene was codon optimized and expressed from a genomic locus in Saccharomyces cerevisiae as a single copy gene under the control of a strong constitutive promoter (TEF1). The gene was introduced into a ‘wild type’ strain and a pdr5 deletion strain (pdr=Pleotropic Drug Resistance gene). The pdr5 gene encodes a pump that has shown to confer a basic level of statin-resistance in Saccharomyces cerevisiae (Hirata & Yano, 1994; Riccardo & Kielland-Brandt, 2011). Furthermore, it has previously been shown that elimination of the pdr5 gene sensitize the strain in question which, in turn, allows for a larger dynamic test range with respect to statin effects.

The efflux pump encoding gene mlcE was integrated into a defined locus of Saccharomyces cerevisiae, CEN.PK 113-11C (MATa MAL2-8C SUC2 his3Δ ura3-52), genome using a yeast expression platform established by Mikkelsen et al. 2012

The yeast strain CEN.PK 113-11C (MATa MAL2-8C SUC2 his3Δ ura3-52) was donated by Dr. Petter Kotter, Institut fur Mikrobiologie, der Johan Wolfgang Goethe-Universität, Frankfurt am Main, Germany. Escherichia coli, DH5□, was used to propagate the plasmids.

The Media

Yeast strains were cultivated in standard liquid yeast peptone dextrose (YPD) or synthetic complete (SC) medium. Yeast transformants were selected on SC medium lacking uracil. Removal of the URA3 marker, via direct repeat recombination, was achieved by growing the strain on SC medium containing 740 mg/L 5-fluororotic acid (5-FOA) and 30 mg/L uracil. The E. coli transformants were selected on LB medium containing 100 μg/mL ampicillin.

The inventors of the present application then tested yeast susceptibility to statins and statin-unrelated compounds by growing yeast strains on solid YPD medium supplemented with compactin, lovastatin, simvastatin, pravastatin sodium, atorvastatin, mycophenolic acid (MPA) or vanillin respectively. Vanillin, MPA and atorvastatin stock solutions were prepared by dissolving the compounds in 99.9% ethanol followed by filter-sterilization. Compactin, lovastatin, and simvastatin were converted to their active forms.

More specifically, the solid compounds were dissolved in 1 mL 99% ethanol, preheated to 50° C., alkalinized with 0.5 mL of 0.6 M NaOH and incubated at 50° C. for 2 hours. The pH of the solutions was then adjusted to 7.2 by adding 0.4 M HCl. Final volume of all the solutions was adjusted to 2 mL with water, resulting in stock solutions of 50 mM. The statin stock solutions were filter-sterilized and stored at −20° C. Compactin and atorvastatin were purchased from Toronto Research Chemicals, lovastatin from Tokyo Chemical Industry, MPA and vanillin from Sigma-Aldrich, and simvastatin was purchased from Ark Pharm.

| | Concentration of the | |
|---|---|---|
| Compound | stock solution [mM] | Source |
| Compactin | 50 | Toronto Research |
| | | Chemicals (Canada, |
| | | Ontario, Toronto) |
| Lovastatin | 50 | Tokyo Chemical Industry |
| | | (Japan, Tokyo) |
| Simvastatin | 50 | Ark Pharm (USA, Illinois, |
| | | Libertyville) |
| Atorvastatin | 10 | Toronto Research |
| | | Chemicals (Canada, |
| | | Ontario, Toronto) |
| Vanillin | 30 | Sigma-Aldrich (USA, |
| | | Missouri, St. Louis) |
| MPA | 320 | Sigma-Aldrich (USA, |
| | | Missouri, St. Louis) |

Plasmid Construction

The mlcE gene was codon-optimized for expression in Saccharomyces cerevisiae (by the company Evolva). The codon-optimized version of the mlcE gene was amplified from plasmid pEN669 (source: Evolva) with primers mlcE-F and mlcE-R. Together with the TEF1 promoter, the amplified gene was cloned into the X-3 vector via USER cloning technique, resulting in plasmid pX3-TEF1-mlcE. To determine the intracellular localization of MlcE, a red fluorescent protein (RFP) was fused to its C-terminus.

For that plasmid pX3-TEF1-mlcE-RFP and a control plasmid pX3-TEF1-RFP were constructed: mlcE without the stop codon was amplified from plasmid pEN669 using primers mlcE-F and mlcE-RFP-R, and RFP was amplified from plasmid pWJ1350 using either RFP-F (for tagging mlcE) or RFP_F+ (for the control plasmid) and RFP_R+ primers. All fragments were amplified by PCR using a USER cloning compatible PfuX7 polymerase.

TABLE
List of plasmids used

| Name | Description | Reference |
|---|---|---|
| pEN669 | mlcE template | Purchased from Evolva |
| PWJ1350 | RFP template | Lisby et al. 2003 |
| pSP-G2 | PGK1, TEF1 template | Partow et al. 2010 |
| pX3 TEF1 mlcE | Plasmid carrying a gene- | This study |
| | targeting cassette for | |
| | expressing mlcE in yeast. | |
| pX3 TEF1- RFP | Plasmid carrying a gene- | This study |
| | targeting cassette for | |
| | expressing RFP-tagged | |
| | mlcE in yeast. | |
| pX3 TEF1 mlcE | Plasmid carrying a gene- | This study |
| RFP | targeting cassette for | |
| | expressing RFP in yeast. | |

Strain Construction

The constructed plasmids were digested with the NotI restriction enzyme (purchased form New England Biolabs), and the linear fragments were used for yeast transformation using the lithium acetate/polyethylene glycol/single carrier DNA transformation method. The URA3 marker in all the constructed strains was excised by direct repeat recombination, and the correct integrations of the gene were verified by colony PCR with one primer annealing in the yeast genome next to the integration site, and one primer annealing inside the introduced DNA.

Targeted deletion of the pleiotropic drug resistance pump (pdr5) encoding gene in the reference and X3: TEFI-mlcE expressing strains was performed, as described by Guldener et al 1996, using the primers PDR5-DEL-F and PDR5-DEL-R.

TABLE
list of strains used

| Name | Genotype | Reference |
|---|---|---|
| CEN.PK113- | MATα MAL2-8C SUC2 his3Δ | Kindly donated by Dr. |
| 11C | ura3-52 | Petter Kötter, Institut |
| | | für Mikrobiologie, der |
| | | Johan Wolfgang Goethe- |
| | | Universität, Frankfurt |
| | | am Main, Germany |
| ARX3 | MATα MAL2-8C SUC2 his3Δ | This study |
| | ura3-52 X3(pTEF1-mlcE) | |
| pdr5Δ | MATα MAL2-8C SUC2 his3Δ | This study |
| | ura3--52 pdr5Δ | |
| AR29pdr5Δ | MATα MAL2-8C SUC2 his3Δ | This study |
| | ura3-52 X3(pTEF1-mlcE) | |
| ARX1 | MATα MAL2-8C SUC2 his3Δ | This study |
| | ura3-52 (pTEF1-mlcE-RFP) | |
| ARX2 | MATα MAL2-8C SUC2 his3Δ | This study |
| | ura3-52 (pTEF1-RFP) | |

### EXAMPLE 2

### Phylogenetic Tree

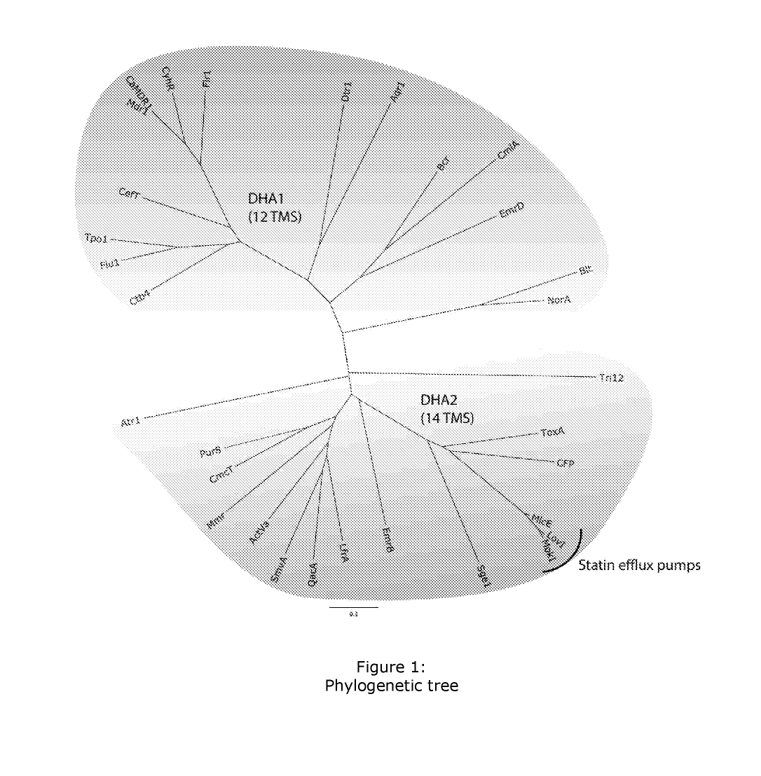

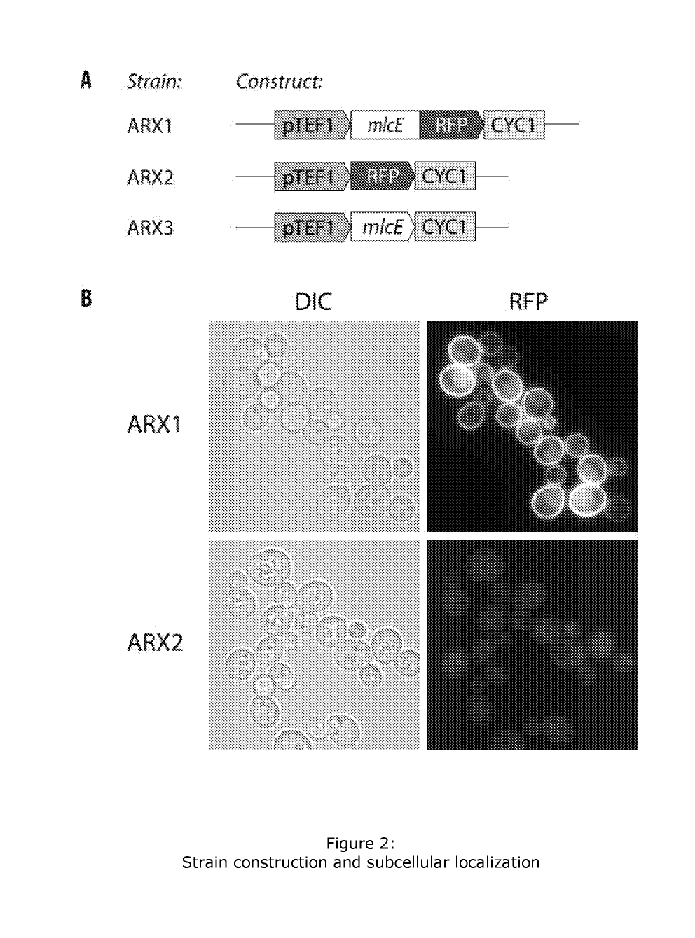

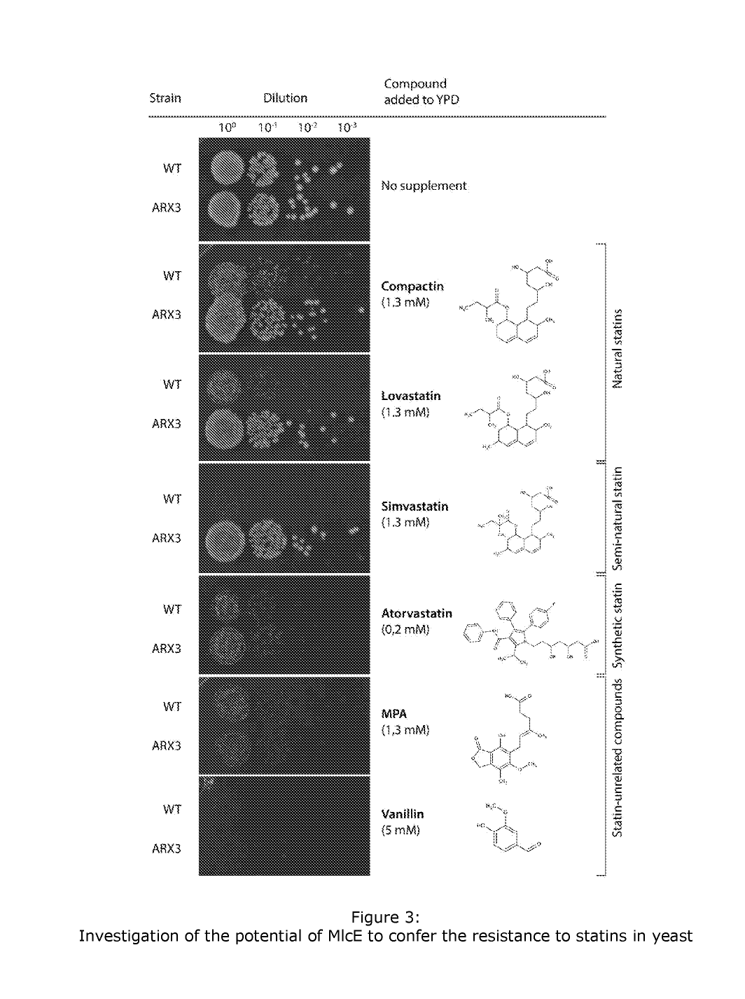

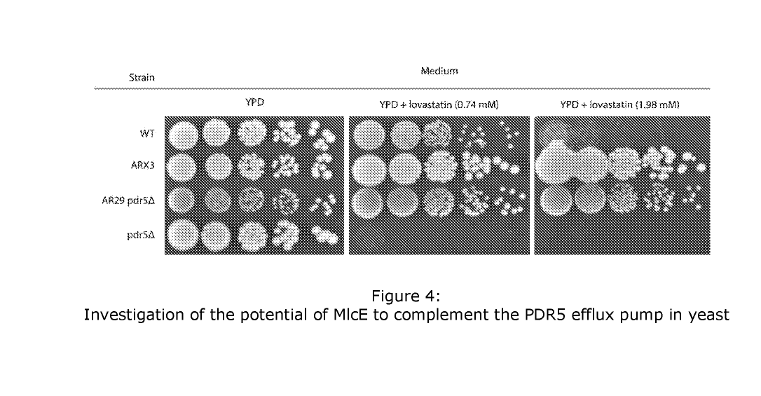

An initial sequence comparison investigation of the putative efflux pump MlcE from the compactin biosynthetic gene cluster using Standard Protein BLAST showed that this protein strongly resembles some of the known export proteins from the major facilitator superfamily (MFS), such as crystal violet efflux pump Sge1 from Saccharomyces cerevisiae and HC-toxin efflux pump ToxA from Cochliobolus carbonum. Moreover, prediction of MlcE topology using the TOPCONS server suggested that MlcE comprises 14 transmembrane-spanning regions (TMS), possibly classifying MlcE to the Drug:H+ antiporter 2 family (DHA2; 14 TMS) of the MFS drug transporters, a family which ToxA and Sge1 belong to as well. The inventors of the present application constructed a phylogenetic tree, which suggests that MlcE, together with its orthologs from the lovastatin and monacolin biosynthetic gene clusters, LovI and MkI, respectively, does indeed belong to the DHA2 family of drug resistance proteins with 14 TMS (FIG. 1).

TABLE
Proteins used for the phylogenetic tree construction

| | | | |
|---|---|---|---|
| | | | Accession |
| | | Representative | Number |
| Protein | Microorganism | Substrate | (UniProtKB) |

MFS-DHA2 (14 TMS)

| | | | |
|---|---|---|---|
| ToxA | Cochliobolus | HC-toxin | Q00357 |
| | carbonum | | |
| Tri12 | Fusarium | Trichotechene | O93842 |
| | sporotrichioides | | |
| CFP | Cercospora kikuchii | Cercosporin | O93886 |
| Atr1 | Saccharomyces | Aminotriazole | P13090 |
| | cerevisiae | | |
| Sge1 | Saccharomyces | Crystal violet | P33335 |
| | cerevisiae | | |
| EmrB | Escherichia coli | CCCP ^{b} | P0AEJ0 |
| LfrA | Mycobacterium | Acriflavin | Q50392 |
| | smegmatis | | |
| QacA | Staphylococcus | Benzalkonium | Q1XG09 |
| | aureus | chloride | |
| SmvA | Salmonella | Ethidium bromide | P37594 |
| | typhimurium | | |
| ActVa 1 | Streptomyces | Actinochodrin | Q53903 |
| | coelicolor | | |
| CmcT | Nocardia | Cephamycin | Q04733 |
| | lactamdurans | | |
| Mmr | Streptomyces | Methylenomycin A | P11545 |
| | coelicolor | | |
| Pur8 | Streptomyces lipmanii | Puromycin | P42670 |

MFS - DHA 1 (12 TMS)

| | | | |
|---|---|---|---|
| Ctb4 | Cercospora nicotinae | Cercosporin | A0ST42 |
| CefT | Acremonium | Cephalosporin | Q8NKG7 |
| | chrysogenum | | |
| Mdr1 | Canidida albicans | Fluconazole | P28873 |
| Flu1 | Candida albicans | Fluconazole | G1UB37 |
| Bcr | Escherichia coli | Bicyclomycin | C6EA15 |
| Blt | Bacillus subtilis | Acriflavin | M1U4Q0 |
| EmrD | Escherichia coli | CCCP ^{b} | P31442 |
| CaMDR1 | Candida albicans | Benomyl | Q9URI2 |
| NorA | Staphylococcus | Acriflavin | P0A0J7 |
| | aureus | | |
| CyhR | Candida maltosa | Cycloheximine | P32071 |
| CmlA | Pseudomonas | Chloramphenicol | Q83V15 |
| | aeruginosa | | |
| Flr1 | Saccharomyces | Fluconazole | P38124 |
| | cerevisiae | | |
| Tpo1 | Saccharomyces | Spermine | Q07824 |
| | cerevisiae | | |
| Dtr1 | Saccharomyces | Dityrosine | P38125 |
| | cerevisiae | | |
| Aqr1 | Saccharomyces | Quinidine | P53943 |
| | cerevisiae | | |

Statin Efflux pumps - unknown family

| | | | |
|---|---|---|---|
| MlcE | Penicillium citrinum | Compactin | Q8J0F3 |
| LovI | Aspergillus terreus | Lovastatin | Q9Y7D4 |
| MkI | Monascus pilosus | Monacolin | Q3S2U5 |

### EXAMPLE 3

### Toxicity Analysis on Dilution Tests

The constructed strains response to different lovastatin levels present in the growth medium were tested using a agar-plate dilution assay (also known as a spot assay). Overnight cultures of the four Saccharomyces cerevisiae strains (wt, ARX3, AR29 pdr5Δ, pdr5Δ,) were diluted to OD₆₀₀ of 0.2 and a fivefold dilution series for each strain was made. Four microliters of each dilution were deposited on a series of agar plates with different concentrations of activated lovastatin (0 mM, 0.74 mM, 1.98 mM). The idea behind this assay is that it allows for reproducible testing of toxic effects by observing at which dilution steps the different strains are able to form visible colonies, under a given concentration of the toxic compound. The growth of the individual strain as a function of time was recorded by photography.

The plate assay (FIG. 4) confirmed that the pdr5Δ, strain is more sensitive to lovastatin than the wild type (wt), as evidenced by the lack of growth even at the lowest tested concentration (0.74 mM). Expression of the mlcE gene allows both the wild type and pdr5Δ, strain to grow at elevated statin concentrations and at the higher dilutions evidencing that the MlcE efflux pump indeed can provide statin resistance in yeast cells, such as Saccharomyces cerevisiae.

### EXAMPLE 4

### Subcellular Localization of the MlcE Efflux Pump)

To determine the subcellular localization of the MlcE efflux pump in Saccharomyces cerevisiae and in order to test the hypothesis that the MlcE protein is in fact a transmembrane efflux pump, the inventors of the present application constructed a C-terminal mRFP fusion and expressed it from the same locus in Saccharomyces cerevisiae. The resulting strain was analyzed by fluorescent microscopy and compared to a Saccharomyces cerevisiae strain that expressed mRFP (cytoplasmic localization) from the same promoter and the same locus as the strain with the RFP-tagged MlcE.

More specifically, the MlcE was tagged with RFP at the C-terminus, and integrated into the previously described site in the yeast genome under the control of TEF1 promoter, resulting in the yeast strain ARX1 (FIG. 2A). Fluorescent microscopy of ARX1 revealed a ring-like distribution of fluorescence around the cell (FIG. 2B top panel), indicating that the tagged putative efflux pump was localized in the plasma membrane. In contrast, the mRFP alone was found to have a uniform cytoplasmic distribution in the control cells ARX2 (FIG. 2B bottom panel), expressing RFP alone from the same locus and controlled by the promoter as in strain ARX1. These results support the prediction that MlcE is a trans-membrane protein and shows that the protein is targeted to the plasma membrane in S. cerevisiae.

Moreover, to determine if the putative efflux pump MlcE has the ability to export statins across the plasma membrane the inventors of the present application also tested whether MlcE confers resistance to statins in yeast. To achieve that, mlcE was expressed from a defined genomic locus in Saccharomyces cerevisiae as a single copy gene under the control of a strong constitutive promoter pTEF1 (FIG. 2A). The resulting yeast strain ARX3 was tested for susceptibility to compactin by serial dilution plating of both wild type (WT) and ARX3 strains on YPD agar plates supplemented with the active form of compactin (FIG. 3). The efflux pump harbouring strain ARX3 showed an increased resistance to compactin present in the medium compared to the wild type strain, suggesting that MlcE is indeed a compactin efflux pump capable of exporting this natural statin out of the cells and not into storage compartments such as the vacuole.

### EXAMPLE 5

### The MlcE Pump in Saccharomyces cerevisiae Confers Resistance Against Other Types of Statins

The inventors of the present application additionally showed that Saccharomyces cerevisiae strains with the inserted transmembrane efflux pump MlcE had an increased resistance not only to compactin but also to the other natural statin, lovastatin, when compared to the wild type yeast strain.

In addition to this, the inventors of the present application surprisingly found that introduction of the transmembrane efflux pump MlcE into Saccharomyces cerevisiae strains also resulted in increased resistance to the semi-natural statin simvastatin, when compared to the wild type yeast strain.

In contrast, MlcE does not seem to have the ability to export compounds, which are structurally unrelated to its natural substrate compactin, namely atorvastatin, vanillin and mycophenolic acid (MPA) because ARX3 strain does not show an increased resistance to these compounds (FIG. 3).

On this basis the inventors of the present application concluded that MlcE is not a multi-drug resistance efflux pump such as for example Pdr5 and Sge1 from Saccharomyces cerevisiae but rather a transmembrane statin specific efflux pump.

In general this means that the efflux pump MlcE provides resistance to both, natural and semi-natural statins, making it a great tool for optimizing yeast cell factories for statin production.

### REFERENCES

(1) Xu W. et al., (2013), “LovG: The Thioesterase Required for Dihydromonacolin”, Angew. Chem. Int. Ed. 2013, 52, 6472-6475.

(2) Abe et al. (2002), “Molecular cloning and characterization of an ML-236B(compactin) biosynthetic gene cluster in Penicillium citrinum” Mol. Genet. Genomics (2002) 267: 636-646.

(3) Hirata D., & Yano K. (1994), “Saccharomyces cerevisiae YDR1, which encodes a member of the ATP-binding cassette (ABC) superfamily, is required for multidrug resistance”, Curr. Genet (1994) 26:285-294.

(4) Riccardo L., & Kielland-Brandt M C. (2011),“Sensitivity to Lovastatin of Saccharomyces cerevisiae Strains Deleted for Pleiotropic Drug Resistance (PDR) Genes”, J Mol Microbiol Biotechnol 2011; 20:191-195.

(5) Hutchinson et al., (2000), “Aspects of the biosynthesis of non-aromatic fungal polyketides by iterative polyketide synthases” Antonie van Leeuwenhoek 78: 287-295.

(6) Mikkelsen et al. (2012), “Microbial production of indolylglucosinolate through engineering of a multi-gene pathway in a versatile yeast expression platform”, Metabolic Engineering 14 (2012) 104-111.

(7) Partow S. et al. (2010), “Characterization of different promoters for designing a new expression vector in Saccharomyces cerevisiae” Yeast 2010; 27: 955-964.

(8) Lisby M. et al. (2003), “Colocalization of multiple DNA double-strand breaks at a single Rad52 repair centre”, Nature Cell Biology, vol. 5, no. 6, June 2003.

(9) Guldener U. et al. (1996), “A new efficient gene disruption cassette for repeated use in budding yeast”, Nucleic Acids Research, 1996, Vol. 24, No. 13 2519-2524.

1. A method for increasing the resistance in a transgenic Saccharomyces cerevisiae to inhibitors of 3-hydroxy-3-methylglutaryl coenzyme A reductase (statins) compared to wild type Saccharomyces cerevisiae, wherein the method comprises overexpressing in the transgenic Saccharomyces cerevisiae one or more polynucleotide(s) of SEQ ID NOs: 1, 2 and/or 3.

2-15. (canceled)

16. The method according to claim 1, wherein the polynucleotide(s) comprise(s) one or more nucleotide variant(s) comprising sequences with a degree of identity to any of SEQ ID NOs: 1, 2 and/or 3 of at least: 80%, at least 85%, at least 90%, at least 95%, at least 97%, at least 98%, or at least 99%.

17. The method according to claim 1, wherein the statin is a natural statin.

18. The method according to claim 17, wherein the natural statin is compactin or lovastatin.

19. The method according to claim 1, wherein the statin is a semi-natural statin.

20. The method according to claim 19, wherein the semi-natural statin is simvastatin.

21. A transgenic Saccharomyces cerevisiae that overexpresses one or more polynucleotide(s) of SEQ ID NOs: 1, 2 and/or 3.

22. The transgenic Saccharomyces cerevisiae according to claim 21, wherein the polynucleotide(s) comprises one or more nucleotide variant(s) comprising sequences with a degree of identity to any of SEQ ID NOs: 1, 2 and/or 3 of at least: 80%, at least 85%, at least 90%, at least 95%, at least 97%, at least 98%, or at least 99%.

23. The transgenic Saccharomyces cerevisiae according to claim 22, wherein one or more of the polynucleotide(s) according to SEQ ID NOs: 1, 2 and/or 3 is/are overexpressed and, wherein one or more of the polynucleotide variants according to claim 22 is/are overexpressed.

24. The transgenic Saccharomyces cerevisiae according to claim 22, wherein the statin is a natural statin.

25. The transgenic Saccharomyces cerevisiae according to claim 24, wherein the natural statin is compactin or lovastatin.

26. The transgenic Saccharomyces cerevisiae according to claim 22, wherein the statin is a semi-natural statin.

27. The transgenic Saccharomyces cerevisiae according to claim 26, wherein the semi-natural statin is simvastatin.
